(19) United States
(12) Patent Application Publication
(10) Pub. No.: US 2009/0054983 A1
(43) Pub. Date: Feb. 26, 2009

(54) BIORESORBABLE BONE IMPLANT

(75) Inventors: Paulus Ignatius J.M. Wuisman, Houten (NL); Martinus Nicolaas Helder, Lelystad (NL)

Correspondence Address: MORRISON & FOERSTER LLP, 12531 HIGH BLUFF DRIVE, SUITE 100, SAN DIEGO, CA, 92130-2040, US

(73) Assignee: TECHNOLOGIESTICHTING STW, Utrecht (NL)

(21) Appl. No.: 11/658,330

(22) PCT Filed: Jul. 25, 2005
(86) PCT No.: PCT/NL2005/000539

§ 371 Date: Sep. 8, 2008

(30) Foreign Application Priority Data
Jul. 23, 2004 (NL) 1026712

(51) Int. Cl. A61F 2/28

(52) U.S. Cl. 623/16.11; 623/23.57

(57) ABSTRACT

The invention relates to an artificial bone implant for preservation, repair, and regeneration of the human musculoskeletal apparatus by means of orthopedic, dental and craniofacial implantation. More in particular, the invention relates to use of osteoinductive compositions comprising stem cells from adipose tissue and osteoconductive calcium phosphates in bioresorbable cages as part of an artificial bone implant for stabilization and renewed alignment of spinal column segments.

### FIELD OF THE INVENTION

The invention relates to an artificial bone implant for preservation, repair and regeneration of the human musculoskeletal system, by means of orthopedic, dental, and craniofacial implantation. More in particular, the invention relates to use of osteoinductive compositions comprising stem cells from adipose tissue and osteoinductive calcium phosphates in bioresorbable poly-L-lactide and poly-L-D-lactide cages as part of an artificial bone implant for stabilization and renewed alignment of spinal column segments.

### BACKGROUND OF THE INVENTION

Instability, spondylolisthesis (shifting or slipping of vertebrae), and degenerative pathology of the lumbar spinal column lead to severe intervertebral disk-related pain in many patients. When conventional treatment such as medication, rest or physiotherapy is not efficient for such patients, lumbar spinal fusion is the generally accepted surgical method for treating these patients. This lumbar spinal fusion, which is referred to as “posterior lumbar interbody fusion” (PLIF) in the field, comprises lumbar spinal column surgery which is carried out from the back of the patient and in which a fusion (ankylosis) of at least two vertebral bodies is realized. The clinical result of PLIF surgery is considered successful if fusion actually occurs. However, in a larger number of patients, this fusion does not occur or occurs to an insufficient extent.

In short, PLIF surgery comprises surgically exposing the respective spinal column segments and correcting the defect of the spinal column, for instance by means of laminectomy (removing the vertebral arch in order to widen the spinal canal), foraminotomy (widening the intervertebral opening), and/or clearing out the intervertebral disk. Optionally, fixation material (screws or plates) is provided during surgery. In order to bring about a fusion between the two vertebral bodies, bone material (spongiosa) is removed from the dorsal part of the pelvic brim by means of separate surgery. This bone material is used in combination with an intervertebral fusion cage is which is generally non-resorbable. The cages may have different compositions, forms and flexibilities. A much used form has an “open box” structure which is filled with the autologous bone (bone of the patient himself or herself), which is optionally mixed with a synthetic carrier material (usually calcium phosphate) and/or a bone growth factor in advance.

The primary purpose of the cages is directed to the recovery of the mechanical properties of the spinal column, i.e. the design of the cages is such that the original space of the intervertebral disk is restored, that the sagittal plane is aligned, and that the bearing capacity of the anterior column is recovered. For this reason, in the conventional procedure, often metal (for instance titanium) cages are used which are sufficiently strong for this function.

However, the stiffness and strength of this type of fusion cages at the same time leads to the occurrence of so-called stress shielding of the implant material inside the cage, a phenomenon in which the implant material experiences no pressure from the spinal column, which adversely affects the rate and extent of bone formation and the subsequent fusion. Stress shielding is one of the reasons that, in many cases, fusions are not successful and PLIF surgery does not succeed.

Another problem which occurs with PLIF surgery is that a significant number of patients (10-50%) experience serious problems because of the removal of the donor bone from the pelvic brim. The problems vary from hematoma and permanent donor morbidity at the site of the bone collection, which is accompanied by a lot of pain, to instability and even fractures of the pelvis. In order to prevent these problems and to increase the success of PLIF surgery, alternative materials for cages and bone transplants are desirable.

Thus, cage materials are currently known, such as the materials PLLA and poly-L/D-lactide (PLDLA; a mixture of poly-L-lactic acid with 30% of poly-D-lactic acid), which are resorbable and which have an elasticity modulus similar to that of vertebral bone. These resorbable cages reduce the occurrence of stress shielding of the transplant material because, with the resorbing of the cage by the body, the newly formed bone material will gradually be loaded more and more.

Various materials and methods have been used for increasing the bone density and the bone volume. Currently, the use of autologous bone is the most reliable method and this bone is an efficient implant material. However, as already said, a drawback is the morbidity at the donor site and the limited availability. The use of allo-implants and xenogeneic bone implants is becoming increasingly more dubious as a result of immunological problems and risk of viral contamination.

The use of only a synthetic carrier material (for instance hydroxyapatite) has the drawback that such a carrier material only has osteoconductive properties and has no osteoinductive properties. Cells which are to provide the fusion of the spinal segments will first need to diffuse from the environment into the scaffold or by supplied into the scaffold by new vessel ingrowth.

The invention now provides a solution to one or more of the above problems.

### SUMMARY OF THE INVENTION

In a first aspect, the present invention provides an artificial bone implant, comprising a resorbable porous matrix in which an osteoinductive composition comprising calcium phosphate and stem cells from adipose tissue is present. Preferably, this porous matrix comprises PLDLA and the calcium phosphate is bicalcium phosphate. The osteoinductive composition may further comprise a growth factor chosen from the group consisting of TGF-βs, BMPs, Osf-1 and LMP-1, preferably Osf-1.

In another aspect, the present invention provides the use of an artificial bone implant according to the invention for bone surgery, particularly spinal fusions.

A further aspect of the present invention relates to a method for preparing an artificial bone implant for use in bone surgery, comprising combining a resorbable porous matrix and an osteoinductive composition comprising calcium phosphate and stem cells from adipose tissue.

In still another aspect, the invention relates to a method for carrying out bone surgery, for instance a lumbar spinal fusion, comprising implanting an artificial bone implant according to the invention into a bone of a patient.

The invention further relates to a method for curing a bone defect by inducing the growth of new bone tissue in a vertebrate comprising removing adipose tissue from a vertebrate and isolating stem cells therefrom, manufacturing an osteoinductive composition by seeding these stem cells on calcium phosphate and introducing the bone implant into the vertebrate by means of a surgical operation.

The invention further relates to a method for replacing cartilage by bone tissue in a vertebrate by inducing the growth of new bone tissue, comprising removing adipose tissue from a vertebrate and isolating stem cells therefrom, manufacturing an osteoinductive composition by seeding these stem cells on calcium phosphate, filling a resorbable porous matrix with the osteoinductive composition to provide an artificial bone implant and introducing this bone implant into the vertebrate by means of a surgical operation.

### DESCRIPTION OF THE FIGURES

FIG. 1. Number of vital cells in fresh cell isolates, divided according to locus (A) and harvesting procedure (B). Vital cells were identified by means of a trypan blue exclusion test. Each point represents an individual isolation. Line: average cell yield within a group. No differences in cell yield between the harvest sites (A; p=0.3; One-way ANOVA) and the different harvesting procedures (B; p=0.69; One-way ANOVA). Abbreviations: AT=adipose tissue; SVF=cell isolate; RS=resection; T-LS=conventional liposuction; US-LS=ultrasound-mediated liposuction.

FIG. 2. Effect of the harvesting procedures on the stem cell frequency in cell isolates. Adipose tissue was harvested by means of resection (RS), conventional liposuction (T-LS), or ultrasound-mediated liposuction (US-LS). Stem cell frequencies were determined using a limiting dilution test, in which scoring was done after 21 days. A group of >10 adhering cells was counted as positive. Frequencies were calculated of that row of culture wells in which fewer than 60% positives were found. A significant difference (p>0.05; independent sample t-test) was detected between the RS and US-LS groups.

FIG. 3. Effect of stimulation of freshly isolated adipose stem cell populations with different doses of rhBMP-2 (ng/ml) for 15 or 30 minutes, followed by 4 days of culture in basal medium, on the mRNA expression of alkaline phosphatase (ALP), osteopontin (OPN), and collagen type I (ColA1). Data are expressed as “fold increase (T/C)”, or increase factor with respect to control, in which “treatment” (T) comprises the rhBMP-2 treatment and “control” (C) is understood to mean the medium without rhBMP-2.

FIG. 4. “Seeding” PKH26 fluorescently labeled cells on BCP® material. Left box, top and bottom: adipose stem cells seeded on BCP®. White arrows indicate individual cells. Center and right box, bottom and top: two different magnifications, where the cells are visible as small globules on the BCP® material; top magnification is after 10 minutes of adhesion, bottom magnification after 30 minutes of adhesion.

FIG. 5. Histological evaluation of adipose stem cell-mediated spinal fusion (4 and 28 days after implantation). (A) PLDLA cage filled with BCP® granules and an adipose stem cell suspension, preceded by implantation. (B) Macroscopic photograph of a sagittal cut through an intervertebral disk between two vertebrae L3 and L4, in which an implant has been placed. (C) PKH26-labeled adipose stem cells in the cage space 4 days after implantation. White arrows indicate individual cells. (D) TRAP/ALP staining of a cut of an implant after 28 days in vivo. TRAP staining is red, and stains the bone-degrading and mineral-degrading osteoclasts (black arrows). ALP staining is blue and indicates bone-forming activity in osteoblasts (black triangles). Newly formed bone is indicated by the white arrows. SC is carrier material.

### DETAILED DESCRIPTION OF THE INVENTION

A “synthetic filler” or “carrier material” as used herein is the—often porous—granular, fiber-like or powdery or at least temporarily mixable or plastic substance or solid substance which has the capability to act as bone-supporting (osteoconductive) adhesion substrate for bone-forming cells or tissues. In the present invention, the “synthetic filler” or the “carrier material” is used in the form of a scaffold (see hereinbelow). In embodiments according to the invention, the osteoconductive carrier material is preferably calcium phosphate.

A “scaffold” or “tissue scaffold” as used herein is the functional description of a synthetic filler or carrier material in its physically supporting or carrying capacity for individual bone-forming cells or bone-forming tissue. It is referred to as a scaffold when the bone-forming cells have adhered to the carrier material and the scaffold preferably has a specific architecture for promoting formation, migration, residence and/or proliferation of bone cells and for providing a structural support during the wound healing.

The terms “fusion cage”, “cage” or “matrix” are alternately used herein and are defined herein as a form-retaining, substantially closed, preferably porous covering or framework with an internal cavity for receiving induction material for bone, i.e. into which the scaffold can be loaded.

In the context of the present invention, the term “porous” is defined as having cavities (pores) of a sufficient dimension to accommodate a cell and to let a cell suspension penetrate through the cavities in the material. The cavities may be the interstitial volume between fibers or particles of the scaffold or the cage or the holes (also called cells) in a substantially homogeneous, open-solid structure.

An “osteoinductive scaffold” as used herein comprises a combination of the osteogenic stem cells from fat and an osteoconductive carrier.

An “osteogenic stem cell” is a stem cell which has been induced to differentiate via the osteogenic pathway, or a stem cell which has the capability to differentiate via the osteogenic pathway as a result of the presence of a recombinant gene coding for a bone growth factor in that stem cell, which gene can be expressed, or a stem cell which is in an environment in which a bone growth factor is present as well in a concentration which leads to induction of differentiation of the stem cell, or a stem cell which has the capability to differentiate via the osteogenic pathway after induction with a bone growth factor.

The expression “repairing a bone defect” as used herein is not limited to actions directed to repairing bone damage or loss of bone, but also to actions directed to creating new bone material at sites where this is desired, as carried out in plastic, reconstructive and esthetic surgery. In particular, the expression is directed to replacing cartilage tissue by bone tissue, as it used in the provision of spinal fusions.

An artificial bone implant according to the invention comprises a resorbable porous matrix or cage. The cage may have any desired form and primarily serves to preserve the shape of the implant and to position the implant in the desired position. Preferably, the cage is a fusion cage as it is used for fusing two vertebrae, but this is not essential. Other cages, for instance those which can be used in bone operations such as cranial surgery, jaw surgery, hip surgery or other bone surgery can also be developed by a skilled person, as long as strength or support can be offered to the osteoinductive composition and positioning with and, if desired also, of the bone elements still present is made possible by means of the cage.

Cage materials in embodiments of the present invention are used in bioresorbable materials. Very suitable are materials such as PLLA or PLDLA; preferably, PLDLA is used. These materials have an elasticity modulus which resembles that of vertebral bone. An additional advantage is that these materials are radiolucent, so that follow-up by means of X-rays and/or CT and MRI scans is well possible. For PLLA and PLDLA, it has been demonstrated that they are resorbed via natural pathways, so that, in the long term, no foreign material remains in the fusion segments.

An example of the use of bioresorbable materials as cage material is that the so-called stress shielding of the implant material, as it occurs in the conventionally used metal cages, occurs to a strongly reduced extent or does not even occur at all. A further advantage of bioresorbable cages, such as PLLA and PLDLA cages, is that they result in increased rates and numbers of bone fusions compared with titanium cages. In addition, these cages have a low immunogenicity.

For manufacturing bioresorbable cages from, for instance, PLLA, lactic acid is converted into dilactide, after which poly-L-lactide (PLLA) can be prepared by esterification and be manufactured into a cage, for instance as described in EP 1 138 285.

In addition to PLLA or PLDLA, optionally, polyglactin 910 (Vicryl) or a different suitable resorbable material may be used as well.

An artificial bone implant according to the invention further comprises an osteoinductive composition comprising calcium phosphate and stem cells.

Of calcium phosphates, it has been demonstrated that they have a good interaction with living bone. Therefore, as osteoconductive carrier material for autologous stem cells, calcium phosphate can very suitably be used. The most extensively studied calcium phosphate configurations are hydroxyapatite (HA) and bi and tricalcium phosphates (BCP and TCP). BCP and TCP are biodegradable, while HA, in particular in the sintered form, is considered non-resorbable. By varying the proportions of BCP, TCP and other components as mentioned hereinbelow, a more or less porous and/or crystalline cement can be manufactured. From biocompatible calcium phosphate scaffold no immunological reactions can be expected.

Preferably, the pores of the scaffold have a diameter of 200-1000 μm, more preferably 400-600 μm. The interconnections preferably have a diameter of 50-200 μm, more preferably 120-150 μm. The ratio of surface to volume is such that a porosity of at least 60%, preferably at least 70%, more preferably at least 80%, still more preferably at least 90% is obtained.

Preferably, a BCP composite (consisting of bicalcium phosphate and 40% hydroxyapatite) is used as a carrier material. More preferably, the BCP BiCalPhOS® (Medtronic Sofamor Danek Memphis, Tenn., USA) is used. The optimization of the choice resides in the fact that, by using this material, a faster resorption/remodeling takes place compared with other carriers.

Other components which can be added to an osteoconductive carrier material are, for instance, biocompatible polymers for an optional increase of the density and strength of the material, substances promoting the adhesion of cells, growth factors and antimicrobial substances.

In the present invention, as stem cells, stem cells from adipose tissue are used. An advantage of the use of stem cells from adipose tissue over the use of stem cells from bone marrow is that, for obtaining the former, prior to the actual spinal column surgery, no separate surgical operation, i.e. a very painful and uncomfortable bone marrow collection, is necessary and neither is a labor-intensive, risky, expensive and time-consuming proliferation of stem cells in the laboratory for in vitro expansion of the bone marrow stem cells.

An artificial bone implant according to the invention therefore comprises an osteoconductive composition comprising stem cells from adipose tissue. There are more and more indications that stem cells present in various tissues have a broad plasticity. Pluripotent stem cells have been identified in embryonic tissue and in adult tissue. However, the use of embryonic stem cells is subject to broad ethical discussions and only small amounts can be obtained.

The development and differentiation potential of stem cells obtained from bone marrow of adult individuals appears to extend beyond the hematopoietic line and comprises the vasculogenic, mesenchymal, non-mesenchymal and endodermal direction. In addition, it was also found that stem cells with broad development possibilities could also have a non-hematopoietic origin, including neurons or muscles. Although the latter have opened up a new field of investigation with a potentially wide range of applications, the role of the bone marrow as a source of stem cells with differentiation possibility to mesenchymal cells and tissues has a longer history. In the past ten years, it has been demonstrated that mesenchymal stem cells (MSC) obtained from bone marrow have the capability to differentiate to chondrocytes, adipocytes, myeloblasts and osteoblasts.

The mesenchymal differentiation potential has led to some successes in a clinical trial of which the purpose was to restore the differentiation in the osteoblast cell line in children with osteogenesis imperfecta and led to further research into MSC-based therapies. However, the most important obstacle for the use of MSC cells obtained from bone marrow is, besides the discomfort for patients, is the low frequency (about 1 MSC per 10⁵ adhering stromal cells), which make cell expansion necessary with all known drawbacks of time, costs and contamination risks and the risk of stem cell differentiation instead of expansion.

It is known that stromal cells from human adipose tissue in vitro exhibit some characteristics of osteoblast differentiation. Just like MSC obtained from bone marrow, cells obtained from adipose tissue have the capability to differentiate to chondrocytes, adipocytes, myeloblasts and osteoblasts if induced with the right cell line-specific induction factors. Adipose tissue stem cells miss many of the above-mentioned drawbacks of cells obtained from bone marrow.

Adipose tissue stem cells can be obtained through resection and from liposuction aspirates (either in a conventional manner or through ultrasound-mediated liposuction), which can be obtained with minimal discomfort, without long purification procedures and with high yield (300 cc aspiration produces a yield of 2 to 6×10⁸ cells). This removes the necessity for in vitro expansion with the associated risks, as it is necessary for bone marrow cells. Preferably, adipose cells are obtained from resection or conventional liposuction is used.

The adipose tissue stem cells can be maintained in vitro for a prolonged time without apparent death and, as autologous stem cells, they are naturally immunocompatible.

The aspiration of adipose tissue (liposuction) can be carried out by use of any method known for that purpose, for instance by means of procedures which have recently been described, with further reference being made to procedures for obtaining stem cells therefrom (Halvorsen et al., 2001, Tissue Eng 7:729-41; Mizuno et al., 2002, Plast Reconstr. Surg. 109:199-209; Zuk et al., 2001 Tissue Eng 7:211-228). The aspirate can be obtained from different parts of the body such as from abdomen, thigh or buttocks. A skilled person will easily be able to determine that the amount and quality of the harvested stem cells can vary according to the site of collection and depth of the aspiration of the adipose tissue. Also, the minimum aspiration volume can be determined to obtain a sufficient amount of stem cells. Aspirates can immediately be processed further, or can be stored for a short time. A skilled person will be able to choose storage conditions such that a sufficient amount and quality of stem cells can be obtained from the aspirate after storage. Preferably, the aspirate is processed immediately.

An osteoinductive composition can then be prepared by combining the calcium phosphate and the stem cells by, for instance, mixing these in a suitable ratio or, for instance, by passive impregnation of the calcium phosphate with suspensions of stem cells. This step is also referred to as seeding stem cells on calcium phosphate. Weight ratios of stem cells:calcium phosphate in the osteoinductive composition are preferably 1:10,000 to 1:10.

An osteoconductive composition may further comprise therapeutic substances such as antibiotics, adhesion substances, growth-promoting substances, antibiotics or bone growth-stimulating proteins (bone growth factors) to induce the stem cells to differentiate via the osteogenic pathway.

Optionally, prior to mixing with the carrier material, the stem cells can be induced to differentiate to osteoblasts. A suitable induction medium which can be used for this is, for instance, Dulbecco's Modified Eagles Medium (DMEM; GibcoBRL-Cat# 11965-084) with 10% Fetal Bovine Serum (FBS; GibcoBRL-Cat# 10437-028), further supplemented by 0.1 mM dexamethasone, 50 mM ascorbate-2-phosphate, 10 mM glycerophosphate, and 1 wt. % antibiotic or antimycotic. The differentiation may, for instance, be carried out with incubation of the stem cells at 37° C. in 5% CO₂ for an optimal number of days still to be determined for this.

Preferably, prior to or after mixing with the carrier material, or even after the filling of the matrix with the osteoinductive composition, the stem cells are induced to differentiate to osteoblasts by use of bone growth factors.

To this end, the bone growth factors may inter alia be added to the osteoinductive composition in order to induce the differentiation of the stem cells to osteoblasts therein. Also, the adipose stem cells can first be brought into contact with bone growth factors, and then be mixed with the carrier material for providing the osteoinductive composition. In alternative embodiments, the induction of the differentiation of the stem cells can be carried out on the bone implant by means of bone growth factors prior to surgical implantation or even after the implant has been introduced into the patient.

Bone growth factors are specific proteins which influence synthesis and degradation processes in the body by regulating cell growth and cell function. The growth factors involved in the formation of bone can be divided into two groups: the bone-inducing and the bone-producing growth factors. The bone-inducing (new bone development in a bone-free environment) growth factors, including the bone morphogenetic proteins (BMPs) can make an undifferentiated stem cell differentiate to a progenitor bone cell (preosteoblast). The bone-producing growth factors, including transforming growth factor β (TGF-β), insulin-like growth factor (IGF), and platelet-derived growth factor (PDGF), can make the number of differentiated bone cells increase in number (proliferate) or differentiate further to a bone matrix-forming cell (osteoblast).

In principle, both types of growth factors can be used in embodiments according to the present invention. Many growth factors as described in various overview publications (Bonewald, 2002, In Bilezikian et al., (eds) Principles of Bone Biology, Academic Press, San Diego, pp. 903-18; Rosen and Wozney, 2002, In Bilezikian et al., (eds) Principles of Bone Biology, Academic Press, San Diego, pp. 919-28; Reddi, 2000, Tissue Eng. 6:351-9; Boden, 2000, Tissue Eng. 6:383-99; Deuel et al., 2002 Arch. Biochem. Biophys. 397:162-172) can be used in embodiments according to the present invention. Preferably, growth factors from the group of TGF-βs (inter alia TGF-β1-5) and BMPs are used. With great preference, one or more of the growth factors of osteoblast stimulating factor-1 (Osf-1), recombinant human BMP-2 (rhBMP-2) and LIM mineralization protein-1 (LMP-1) are used. With still greater preference, Osf-1 (also known as HB-GAM, pleiotrophin) is used. This factor, an 18-kDa cytokine which monitors various functions, ensures fast and strong adhesion of the Osf-1-transduced stem cells to the surface of the carrier material, so that the osteoblast differentiation is accelerated. In addition, Osf-1 recruits osteoblasts from the environment of the implant and it promotes the angiogenesis in order to ensure improved rates of repair.

Growth factors can be obtained in two manners. One method is the extraction from tissues or tissue fluids (autologous, allologous or xenologous), the other is synthesizing by means of gene transcription of modified DNA in rapidly dividing cells in culture (recombinant techniques). An example of the first method is PRP (platelet-rich plasma), in which the body's own growth factors (mainly PDGF and TGF-β) are obtained from blood.

The use of an osteogenic growth factor at the site of the implant to induce fusion can lead to high diffusion rates and short half-life periods of the osteogenic factor, with associated short periods that threshold values of the osteogenic factor are maintained at the site where the induction is to take place. In addition, as a result of diffusing away the growth factor, bone formation can even take place outside the fusion area. Also with this form of fusion induction, cells which are to provide the fusion of the spinal segments will first need to diffuse from the environment into the scaffold or be supplied into the scaffold by vessel ingrowth. The chance of this becomes smaller as the biological half-life periods are shorter and/or diffusion rates are higher. Another disadvantage is that (recombinant) osteogenic factors are expensive and can give rise to sensibilization.

It is possible to avoid the use of the above-mentioned very expensive growth factors by, for instance, using the temporary or non-temporary expression of a recombinant introduced gene coding for an osteogenic factor. Therefore, preferably a stem cell is formed which is able to produce the relevant growth factor by itself, for instance as described in EP 0 890 639 and US 2002102728. To this end, stem cells may, for instance, be transfected with adenoviral vectors into which the relevant genes for osteogenic growth factors have been introduced and which vectors can be expressed in the stem cells.

For preparing an artificial bone implant according to the invention, a resorbable porous matrix can very suitably be combined with an osteoinductive composition as described hereinabove to provide an artificial bone implant according to the invention. Thus, the porous matrix can very suitably be combined with an osteoinductive composition by means of diffusion, electrophoresis, centrifugal forces or cross-linking.

An artificial bone implant according to the invention may optionally be processed further after manufacturing thereof, for instance by grinding, polishing, providing attachment possibilities such as screw holes or hooks, or crushing.

An artificial bone implant according to the invention may have a complex geometrical shape. The shape may have, for instance, cuts, internal cavities, recesses or protrusions. The bone implant may be shaped such that a specific bone or specific bones or parts thereof or spaces between bones or cavities in bones can be replaced or filled therewith. The bone implant can be dimensioned and shaped prior to surgery specially for a particular patient. Also, the bone implant may have a simple shape, such as a block, which is intended to be shaped by a surgeon during a surgical operation.

The bone implant can be made to fit for the bone defect and may be tapered or finished with oblique sides or be provided with engaging parts for enhancing the fit.

An artificial bone implant according to the invention in which stem cells obtained from adipose tissue are used offers a number of attractive advantages. From a medical point of view, the advantages of this new bone implant and the associated application possibilities include:

(i) a one-step procedure for the patient

(ii) no important changes in the surgical procedure itself for the patient

(iii) use of resorbable x-ray-permeable cage material, without leaving foreign material in the body in the long term

(iv) only inert resorbable carrier material is placed in the cage, which can be produced unlimitedly according to reproducible, verifiable industrial standards applicable to medical use

(v) replacement of autotransplant by autologous stem cell preparations, obtained through minimally invasive techniques during the same surgical operation, while the adverse side effects associated with harvesting pelvic brim bone transplant are prevented.

(vi) autologous stem cells are naturally immunocompatible and there are no ethical issues associated with their use, in contrast with the use of embryonic stem cells.

From an economic point of view, the advantages include:

(vii) one-step surgery can be carried out, whereas the use of bone marrow-obtained stem cells usually requires bone marrow aspiration in advance, which entails several visits to the clinic. In addition, expensive bone marrow cell purification and proliferation techniques (soaking) are necessary for the actual spinal column surgery.

(viii) surgical time will not be prolonged, but possibly even reduced

(ix) the use of very expensive growth factors can be avoided.

A bone-surgical method according to the invention comprises any method for repairing a bone defect in a vertebrate, preferably a lumbar spinal fusion.

A bone-surgical method according to the invention comprises the step of removing adipose tissue from a vertebrate, for instance by means of liposuction as described hereinabove.

Further, a bone-surgical method according to the invention comprises the step of isolating stem cells therefrom. Such methods are known to a skilled person (see inter alia Halvorsen et al., 2001, Tissue Eng 7:729-41; Mizuno et al., 2002, Plast Reconstr. Surg. 109:199-209; Zuk et al., 2001 Tissue Eng 7:211-228).

After the stem cells have been obtained from the adipose tissue, by the induction of the differentiation of these stem cells to osteoblasts by induction media intended for that purpose, a population of osteogenic cells can be obtained. The purpose of the induction is to induce the stem cells to develop via the osteogenic pathway, i.e. the differentiation from stem cell or progenitor bone cell (osteoprogenitor cell) to osteoblast. Based on in vitro bone formation, the process of the development of osteoprogenitor cells from a stem cell stage to a completely functional bone matrix-synthesizing osteoblast is divided into three stages: 1) proliferation, 2) extracellular bone matrix development and maturation, and 3) mineralization.

A bone-surgical method according to the invention subsequently comprises the step of manufacturing an osteoinductive composition by seeding stem cells on calcium phosphate, and filling a resorbable porous matrix with this osteoinductive composition to provide an artificial bone implant. The surgical method finally comprises the introduction of the bone implant into the vertebrate by means of a surgical operation.

Inducing the cells to differentiate may, in principle, be done at any given moment. Preferably, the differentiation induction step is carried out before seeding the stem cells on the carrier material.

The use of the transplant according to the invention will lead to faster recovery of functionality of the spinal column without the drawbacks associated with autologous bone implants.

Further, the invention makes a one-step surgical procedure possible. This is because, as a result the use of stem cells obtained from adipose tissue, much higher yields of stem cells can be obtained compared with stem cells obtained from bone marrow. As a result, it is not necessary to expand the stem cells by means of laboratory culture. The liposuction step can be carried out within the PLIF surgery procedure, and the extraction of stem cells therefrom, the induction to differentiate and the seeding on calcium phosphate can be carried out within 1-1.5 hours.

An advantage of the method and use according to the invention is that the existing steps can be maintained in the performance of the surgery so that no important changes need to be made in the surgical procedure. In addition, the one-step procedure saves the patient a second hospitalization and surgery and has great personal (for the patient) and socioeconomic advantages. Further, no ethical problem is expected due to use of adult stem cells and no expensive growth factors are needed.

The methods and materials of the present invention can be used in all fields in which bone transplantation is necessary, for instance in orthopedic and neurosurgical fields, but also in dentistry. In general, the methods and materials of the invention can be used to repair a bone defect as a result of, for instance, trauma, tumor, infection, and loose joint implants.

The use of the method and the materials of the present invention is particularly advantageous in those cases in which it is desirable to induce bone growth at those sites where the cartilage present causes problems, and where the bone needs to be take over the function of the cartilage. This is most importantly used in making spinal fusions, i.e. ankylosing vertebrae.

### EXAMPLES

### Example 1

### Isolation of Stem Cells from Different Fat Depots, and the Effect of the Harvesting Procedure Used

Adipose tissue is normally available in large quantities and can be obtained through different surgical operations. The three most commonly used methods are en-bloc resection, vacuum or conventional liposuction, and ultrasound-mediated (high-frequency vibration-mediated) liposuction (Illouz, 1983, 1984; Dolsky, 1984; Scuderi, 1987; Topaz, 2001; Miller, 1991; Suslick, 1990). The three surgical procedures result in a minimal burden for the patient and few complications at the site of fat harvest. We have tested whether the yield and growth characteristics of the adipose stem cells are influenced by the harvest site and the method of isolation.

Fat was harvested from different regions: the abdomen, hip and thigh, back and breast (method according to Zuk et al, 2001). A trypan blue exclusion test was used to determine the number of vital cells per procedure, and this was related to both the harvest site and the harvesting procedure used. It was found that 81±2% (average±standard deviation of the average (SEM)) of the cells were vital. The cell yield was similar at the different harvest sites: Abdomen 0.7×10⁶±0.1×10⁶ cells/gram of adipose tissue (average±SEM, n=16), hip and thigh 0.5×10⁶±0.07×106 cells/g (average±SEM, n=11), back 1.1×10⁶±0.3×10⁶ cells/g (average±SEM, n=2), and breast fat 0.6×10⁶±0.3×10⁶ cells/g (average±SEM, n=4) (FIG. 1A). There were no differences between the different harvesting procedures (tested in hip/thigh and abdomen regions). The yields of vital cells per gram of adipose tissue were 0.7×10⁶±0.1×10⁶ cells, (average±SEM, n=13) for en-bloc resection, 0.5×10⁶±0.1×10⁶ cells/g (average±SEM, n=8) for conventional liposuction, and 0.6×10⁶±0.2×10⁶ cells/g (average±SEM, n=6) for ultrasound-mediated liposuction (FIG. 1B).

Limiting dilution tests were used to determine the stem cell frequency within the isolated cell fraction under the influence of the isolation method. It was found that, within the isolated cell fraction of resected fat, 6.3±1.7% (average±SEM, n=8) of the cells exhibited stem cell properties (adhesion and clonal proliferation), while, in the case of conventional liposuction and ultrasound-mediated liposuction, this was 2.9±1.9% and 0.4±0.2%, respectively (average±SEM, n=4 and n=5, respectively) (FIG. 2). The doubling time of the populations in the logarithmic growth phase was 2.4±0.34 days for fat obtained through resection (average±SEM, n=4), 2.8±0.64 days (average±SEM, n=4) for fat harvested through conventional liposuction, and no fewer than 26.1±4.6 days (average±SEM, n=4) for ultrasound-mediated liposuction (significantly lower than the two other methods (p=0.007 for both)).

### Example 2

### Brief (15-30 Minutes) Stimulation of Freshly Isolated Adipose Stem Cells with Recombinant Human BMP-2 Results in Induction of the Osteogenic Phenotype

Fat obtained through resection was processed as described hereinabove, and the stem cell isolate was stimulated for 15 or 30 minutes with different doses (1-1000 ng/ml) of recombinant human bone morphogenetic protein-2 (rhBMP-2) in DMEM medium supplemented by 10% Fetal Calf Serum (FCS), 500 μg/ml streptomycin, 600 μg/ml penicillin, 50 μg/ml gentamycin, 2.5 μg/ml amphotericin B, 0.1 mg/ml vitamin C (vit. C), and 10 mM β-glycerophosphate (β-GP). As a control, the same medium, but without rhBMP-2, was included. Then, the media were removed, and the cells were cultured for 4 days in the above-described medium, but without BMP-2, vit. C, and β-GP. After 4 days, total RNA was isolated from the cells and cDNA was synthesized according to standard methods (Trizol® procedure and cDNA synthesis using Transcriptor® reverse transcriptase and random primers, respectively), and the method of real time reverse transcriptase-polymerase chain reaction (RT-PCR) was used to quantify the mRNA expression levels of the osteogenic markers alkaline phosphatase (ALP), osteopontin (OPN) and collagen type I (col1A1) with the aid of a LightCycler® (Roche). As a control household gene, 18S was used, to which the values of the osteogenic markers were normalized. The results thereof are shown in FIG. 3 as “treatment over control” (T/C) values in which “treatment” comprises the rhBMP-2 treatment and “control” is understood to mean the medium without rhBMP-2.

It appears that these very brief stimulations with rhBMP-2 of freshly isolated stem cell preparations are already sufficient to induce the osteogenic phenotype (as characterized by the up to >27-fold increase of the mRNA expression of the osteogenic markers shown), where the optimal rhBMP-2 dose appears to be between the (physiological values) 1 and 10 ng/ml.

These experiments show that a very brief stimulation of freshly isolated stem cell preparations with physiological doses of rhBMP-2 can already result in induction to the osteogenic phenotype, which means that osteogenic stimulation can be fitted into a one-step surgical procedure.

### Example 3

### “Seeding” Stem Cell Preparations in a BCP® Carrier Material

Adipose stem cells (either freshly isolated or cultured) were labeled with the fluorescent dye PKH26® (Sigma) according to the method described by the manufacturer. The fluorescently labeled cells were then suspended in PBS, to a density of 1-1.5×10⁶ cells/ml. Of this suspension, 0.5 ml was dropped on specially made Biphasic Calcium Phosphate (BCP®) disks (Ø1.4 cm, 4 mm high; Medtronic-Bakken Research Center, Maastricht) in 12-well plates, and incubated for variable times to determine the adhesion times. The PKH26 label, which accumulates in the membranes of the cells, makes it possible to use a direct evaluation method to analyze the cellular distribution after fixation and embedding in Methyl Methacrylate (MMA; a plastic embedding agent) cuts. In addition, BCP® disks were fixed, dried, and analyzed for the presence and distribution of the cells by means of scanning electron microscopy (SEM). An example of these analyses is shown in FIG. 4.

It appears that, due to capillary action, cells distribute very homogeneously through the BCP® material, and that adhesion can take place within 10 min.

### Example 4

In vivo Spinal Fusion Using Bioresorbable Polylactide (PLDLA) Cages, BCP® Carrier Material, and Freshly Isolated Stem Cell Preparations in a One-Step Surgical Procedure in an Animal Model, the Goat

Per goat, the one-step surgical procedure was carried out as follows. Adipose tissue (40-80 grams) was collected around the kidney, after making an incision at the height of the lumbar spinal column. The adipose tissue was then processed as described hereinabove. During this processing period, the spinal fusion procedure (posterior lumbar interbody fusion; in this case removal of the intervertebral disk between the vertebrae L3 and L4 followed by implantation of a PLDLA cage filled with BCP® granules into which adipose stem cells have been seeded) was prepared further until right before the cage implantation stage, in other words, up to and including making the implantation tunnel through which the cage is put in the right place.

After processing of the stem cell preparation, two million cells were labeled with PKH26 (see Example 3), and were resuspended in 1.5 ml of serum-free medium after washing (in rhBMP-2 stimulation, after the PKH26 labeling, a 15-minute incubation step with 10 ng/ml rhBMP-2 can be introduced). For the preparation of the bioresorbable cage construction, a thin layer of freshly taken blood was introduced into a cage, which provided a sealing on the bottom side due to clotting. Subsequently, the cage was filled with BCP® granules, and the cell suspension was dropped into the cage. Initial adhesion was brought about by leaving the construct untouched for a minimum of 45 minutes (FIG. 5A). After “topping off” the construct by dropping a layer of blood and letting it clot in the top side of the PLDLA cage, the spinal fusion procedure was completed by implantation of the cage/BCP®/stem cell construct into the implantation tunnel prepared for this, after which the wound was closed. The total procedure, from the first incision to implantation, took approx. 3 hours in this series of experiments, but this can probably be reduced in the future by practice and computerization of parts of the procedure. The goats were sacrificed after either 4 days (to guarantee visualization of the PKH26-labeled cells) or after 28 days (to visualize initial bone formation). The lower backs were dissected, x-ray photographs were taken, and the fused spinal segment (intervertebral disk L3-L4+half of vertebrae L3 and L34 themselves) was sawed into various sagittal slices, which were photographed and processed for histology. In the 4-day samples, the presence of the labeled stem cells could be verified (FIG. 5B), and thus the feasibility of the one-step surgical scheme. It was thereby shown that it is possible for adipose stem cells to be harvested, processed, and (whether labeled or unlabeled) be introduced into the cage, where they are demonstrable to be vital and at the site for, in any case, 4 days.

In the 28-day samples, the PKH26-fluorescence on the stem cells was found to be no longer detectable. What was possible at this time was to look at bone formation and bone degradation by means of a Goldner staining and a combined alkaline phosphatase (ALP)/tartrate-resistant acid phosphatase (TRAP) staining. Here, both a high initial bone-forming activity (characterized by inter alia a high alkaline phosphatase activity and bone-osteoid formation) and a high BCP® carrier material-degrading activity were observable (visualized by a high number of TRAP-positive bone and mineral-resorbing cells, the polynuclear osteoclasts). So, it appears from this that it is possible to replace the cartilage originally present and to induce bone formation on site.

1. An artificial bone implant, comprising a resorbable porous matrix further comprising an osteoinductive composition said composition comprising calcium phosphate and stem cells from adipose tissue.

2. The implant of claim 1, wherein said porous matrix comprises PLDLA.

3. The implant of claim 1, wherein said porous matrix consists of PLDLA.

4. The implant of claim 1, wherein said calcium phosphate is bicalcium phosphate.

5. The implant of claim 1, wherein said composition further comprises at least one growth factor chosen from the group consisting of one or more TGF-βs, one or more BMPs, Osf-1 and LMP-1.

6. The implant of claim 5, wherein said growth factor is Osf-1.

7. The implant of claim 1, with a porosity of at least 80%.

8. The implant of claim 1, wherein the ratio of resorbable porous matrix:osteoinductive composition is in the range from about 1:10 to 10:1.

9. The implant of claim 1, wherein the ratio of stem cells from adipose tissue:calcium phosphate in said osteoinductive composition is in the range from about 1:10,000 to 1:10.

10. An artificial bone implant, which comprises adipose cells obtainable by resection or conventional liposuction of adipose tissue.

11. (canceled)

12. (canceled)

13. An osteoinductive composition comprising calcium phosphate and stem cells from adipose tissue.

14. A method for preparing an artificial bone implant of claim 1, which method comprises combining a resorbable porous matrix with an osteoconductive composition comprising calcium phosphate and stem cells from adipose tissue.

15. A method of bone surgery in a subject, comprising implanting an artificial bone implant according to claim 1 into a bone of a subject in need of such surgery.

16. A method for healing a bone defect in a vertebrate subject by inducing new bone tissue, which method comprises surgically introducing a bone implant into said vertebrate subject which implant is obtainable by filling a resorbable porous matrix with an osteoinductive composition prepared by seeding stem cells on calcium phosphate.

17. The method of claim 16, wherein said surgery is lumbar spinal fusion.

18. A method for replacing cartilage by bone tissue in a subject by inducing new bone tissue, which method comprises and surgically introducing a bone implant into said vertebrate subject which implant is obtainable by filling a resorbable porous matrix with an osteoinductive composition prepared by seeding stem cells on calcium phosphate.
